(19) United States
(12) Patent Application Publication
(10) Pub. No.: US 2010/0298560 A1
(43) Pub. Date: Nov. 25, 2010

(54) PROCESS FOR PREPARING MYCOPHENOLATE MOFETIL

(75) Inventors: Byoung Taek Choi, Seoul (KR); Yun Bum Ham, Gyeonggi-do (KR); Yong Kyu Park, Gyeonggi-do (KR); Sung Hun Bang, Chungcheongbuk-do (KR); Jin Woong Kim, Gyeonggi-do (KR); Seung Uk Lee, Seoul (KR); Hyun Jun Jung, Gyeonggi-do (KR)

Correspondence Address: FROMMER LAWRENCE & HAUG, 745 FIFTH AVENUE- 10TH FL., NEW YORK, NY, 10151, US

(73) Assignee: CKD Bio Corp., Seoul (KR)

(21) Appl. No.: 12/863,569

(22) PCT Filed: Jan. 8, 2009
(86) PCT No.: PCT/KR09/00086

§ 371 Date: Jul. 19, 2010

(30) Foreign Application Priority Data
Feb. 1, 2008 (KR) 10-2008-0010709

(51) Int. Cl. C07D 413/12; C07D 307/88

(52) U.S. Cl. 544/153; 549/469

(57) ABSTRACT

The present invention relates to an improved method of manufacturing mycophenolate mofetil. More particularly, the present invention relates to a method of manufacturing mycophenolate mofetil with high purity comprising : a) converting mycophenolate to an amine salt by reacting with an amine base; and b) reacting the resultant with a halogenating agent and 2-morpholinoethanol continuously.

### TECHNICAL FIELD

The present invention relates to an improved method of manufacturing mycophenolate mofetil represented by the following formula 1. More particularly, the present invention relates to a method of manufacturing mycophenolate mofetil with high purity represented by the following formula 1 comprising : a) converting mycophenolate represented by the following formula 2 to an amine salt represented by the following formula 3 by reacting with an amine base; and b) reacting the resultant with a halogenating agent and 2-morpholinoethanol continuously,

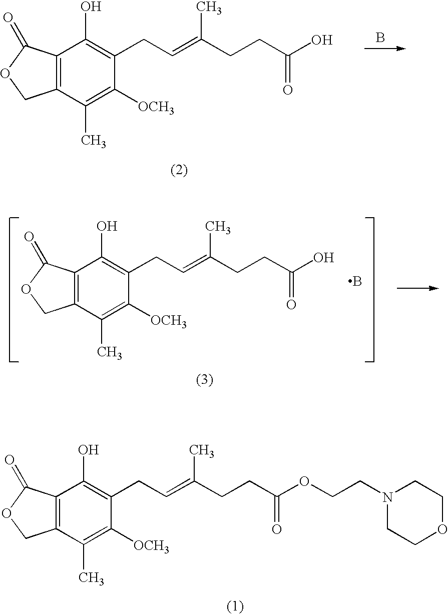

wherein, in the above reaction, B represents an aliphatic or aromatic amine base.

### BACKGROUND ART

The mycophenolate mofetil (MMF) represented by the above formula 1 is an immuno-suppressant commercially available as CellCept™. It has been recently shown very effective in the treatment of systemic lupus erythematosus (SLE), which has not been improved when treated by other immuno-suppressants, and is thus widely used as an immuno-suppressant to prevent lupus nephritis and other symptoms.

U.S. Pat. No. 4,753,935 discloses a general method to manufacture mycophenolate mofetil (MMF). According to this patent, there are two standard esterification methods to manufacture the MMF. One method is to manufacture it via esterification with mycophenolic acid chloride and 2-morpholinoethanol, and another method it to manufacture it via condensation reaction of mycophenolic acid and 2-morpholinoethanol by using dicyclohexylcarboimide (DCC).

That is, in the synthesis of MMF by the reaction with mycophenolic acid chloride and 2-morpholinoethanol, mycophenolic acid chloride was manufactured from mycophenolic acid by using chlorinating agent followed by reaction with 3 equivalents of 2-morpholinoethanol. However, this method has a drawback that it generates dimers and other impurities which are difficult to remove.

In the synthesis of MMF by condensation by using DCC, there is also a drawback that it generates urea and other impurities which are difficult to remove. Further, the color of the target product is violet and thus it is necessary to discolor the target product by further purification to obtain a commercially acceptable white colored product.

Meanwhile, as alternatives, U.S. Pat. No. 5,247,083 discloses a method of azeotropic removal of water; WO 00/34503 discloses a method via enzymatic catalytic reaction; and WO 04/089946 discloses a method using microwave. However, the above methods are not suitable for industrial application due to problems such as low yield, change of color into violet after reaction, etc.

A method known so far suitable for the manufacture of MMF is disclosed in U.S. Pat. No. 4,753,935, where mycophenolic acid is halogenated and then esterified. However, this method, as mentioned above, has a drawback of generating dimers and other impurities and also requires improvement in color of the final product. Therefore, various efforts have been made to resolve the above problems of dimers and other impurities which are difficult to remove. WO 05/023791 and WO 05/105769 disclose a method to remove dimers and other impurities generated during purification as post-treatment after the manufacture of MMF, or a method to remove dimers during the manufacturing process, by using a catalyst such as CAS, PTSA, FeCl₃, CaCl₂. However, the above method is not also suitable for industrial application because of low production yield, high manufacturing cost due to requirement of additional purification process, and impurities in the catalyst itself. Accordingly, there has been an urgent need for the development of an improved method for the manufacture of MMF with high yield and high purity not necessitating a further purification process by minimizing the production of dimers or impurities by performing one-pot reaction.

### DISCLOSURE OF INVENTION

Technical Problem

An object of the present invention is to provide a method of manufacturing mycophenolate mofetil (MMF) represented by the above formula 1 with high purity and high yield by fundamentally blocking the generation of dimers and other impurities that may be generally produced during halogenation process by synthesizing amine salt of mycophenolate as an intermediate in the course of manufacturing MMF represented by the above formula 1 by halogenation of mycophenolic acid and its esterification with 2-morpholinoethanol.

### TECHNICAL SOLUTION

The present invention relates to a method of manufacturing mycophenolate mofetil comprising:

(a) synthesizing an amine salt of mycophenolate represented by the Formula 3 below by reacting mycophenolic acid represented by the Formula 2 below with a C₁-C₁₂ aliphatic or aromatic amine base; and

(b) manufacturing mycophenolate mofetil represented by the Formula 1 below by halogenation of the amine salt of mycophenolate represented by the Formula 3 below and esterification with 2-morpholinoethanol

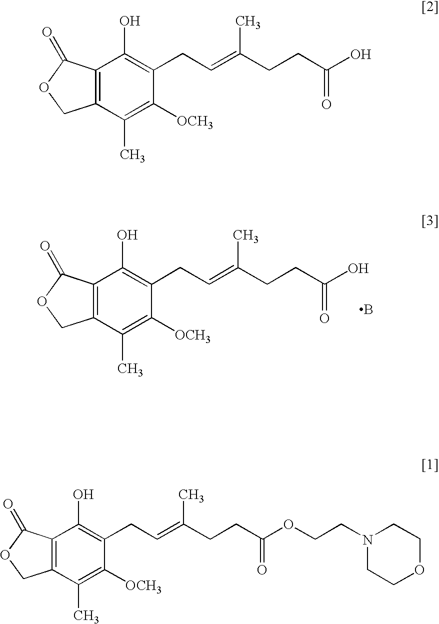

wherein, in the above Formula 3, B is an aliphatic or aromatic amine base.

Further, the present invention also relates to a method of manufacturing mycophenolate mofetil (MMF) represented by the above formula 1 in white color with purity of 99.8% or higher by continuously conducting acidification and alkalinization for the above reaction product as post-treatment processes, and adding an alkali metal sulfite-based compound to the resulting acidified reactant, thereby preventing discoloration without additional purification step.

### ADVANTAGEOUS EFFECTS

In the manufacturing process according to the present invention, the generation of dimers, which are normally produced during halogenation of mycophenolic acid, are fundamentally blocked and other impurities produced thereof are also minimized by introducing an amine salt of mycophenolic acid represented by the above formula 3 as an intermediate for the manufacture of mycophenolate mofetil (MMF) represented by the above formula 1, and thus a commercially acceptable level of MMF with high purity and high yield can be obtained.

Further, in the manufacturing process according to the present invention, acidification and alkalinization of MMF as post-treatment processes are conducted continuously to improve its color and remove other impurities present in small amounts. Besides, in acidification process, a small amount of an alkali metal sulfite-based compound is added as a discolorant thereby obtaining MMF represented by the above formula 1 with high purity and improved color in whiteness not necessitating additional discoloration and purification.

### BRIEF DESCRIPTION OF DRAWINGS

FIG. 1 shows the result of HPLC preformed for the reaction mixture used in the manufacturing method of mycophenolate mofetil (MMF) according to the present invention.

FIG. 2 shows the result of HPLC preformed for the reaction mixture used in the manufacturing method of mycophenolate mofetil (MMF) according to the method disclosed in U.S. Pat. No. 4,753,935.

FIG. 3 shows the result of HPLC preformed for the white-colored compound obtained as a result of post-treatment of the reaction mixture used in the manufacturing method of mycophenolate mofetil (MMF) according to the present invention.

### BEST MODE FOR CARRYING OUT THE INVENTION

With respect to halogenation of carboxylic acid, generally known in the art, it is very natural that an excess amount of or an equal amount as a solvent of a halogenating agent is used relative to the amount of carboxylic acid, and therefore, after the reaction, an excess halogenating agent is removed by evaporation under reduced pressure in an anhydrous condition, and the acyl halide remnant is dissolved in an inert solvent and added dropwisely to a secondary reactant.

However, as mentioned above, the acidity of a halogenating agent and its excess use result in generation of various impurities of dimers including those produced by halogenation of aromatic hydroxyl group and those produced by two molecules of mycophenolic acid, and they are also very difficult to remove.

In the present invention, in order to resolve the production of dimers fundamentally, mycophenolic acid was reacted with various amine bases to obtain a novel amine salt of mycophenolate, and then by performing a series of subsequent steps MMF was finally manufactured. As a result, dimers were not produced at all and also the amounts of various impurities were greatly reduced thus enabling MMF with more than 99.8% purity. Further, the amount of a halogenating agent to be used was greatly reduced. For example, an excess amount of a halogenating agent of greater than 3 equivalents was used in the conventional halogenation of carboxylic acid while only 1-1.2 equivalents of a halogenating agent is used in the present invention to obtain the target compound. Therefore, the present invention, with its cost effectiveness in production and environment-friendliness, is of great industrial applicability.

To date, there has never been a method disclosed on manufacturing high purity MMF by using an amine salt compound represented by the above formula 3 as an intermediate. In addition, in the manufacture of MMF, halogenation has never been successfully completed by using an amine compound as an intermediate with a halogenating agent in a stoichiometric amount.

In other words, the manufacturing method according to the present invention is an industrially useful one clearly distinguished from the conventional ones in that it can fundamentally block the generation of dimers, which have been difficult to remove by using the known methods, and minimize the production of other impurities by stabilizing the reactivity to other reaction sites, which may produce those impurities, thereby producing a target product with high purity and high yield at once.

The superiorities of the present invention can be further confirmed by conducting comparative experiments with a conventional method disclosed in U.S. Pat. No. 4,753,935.

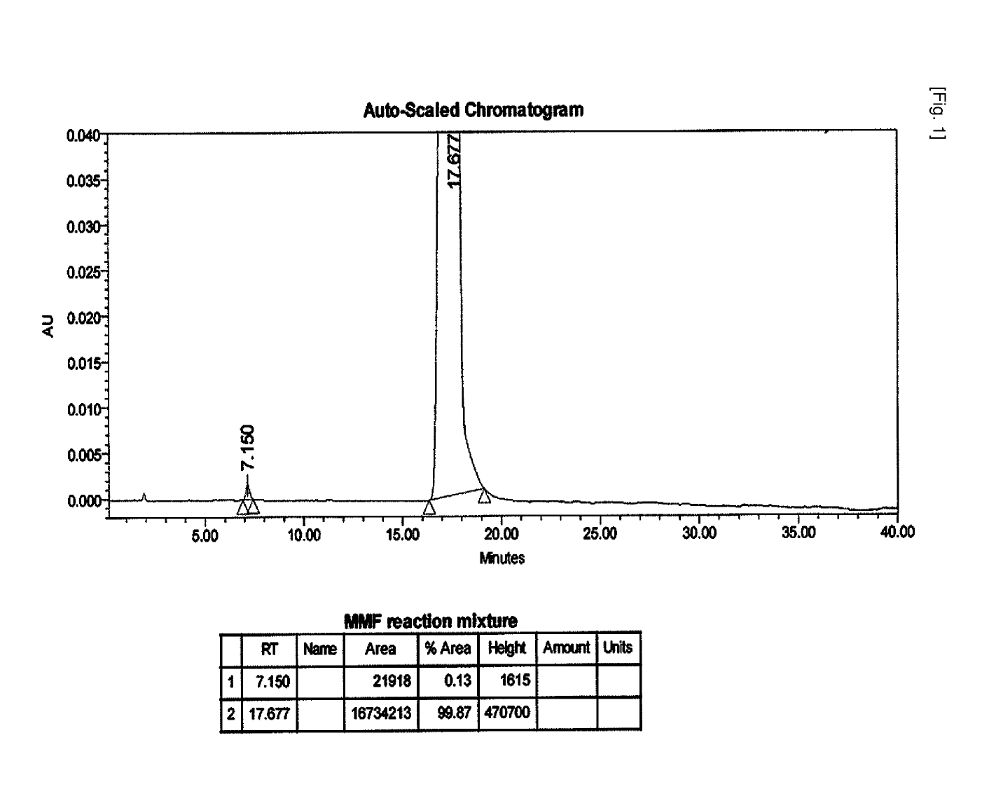

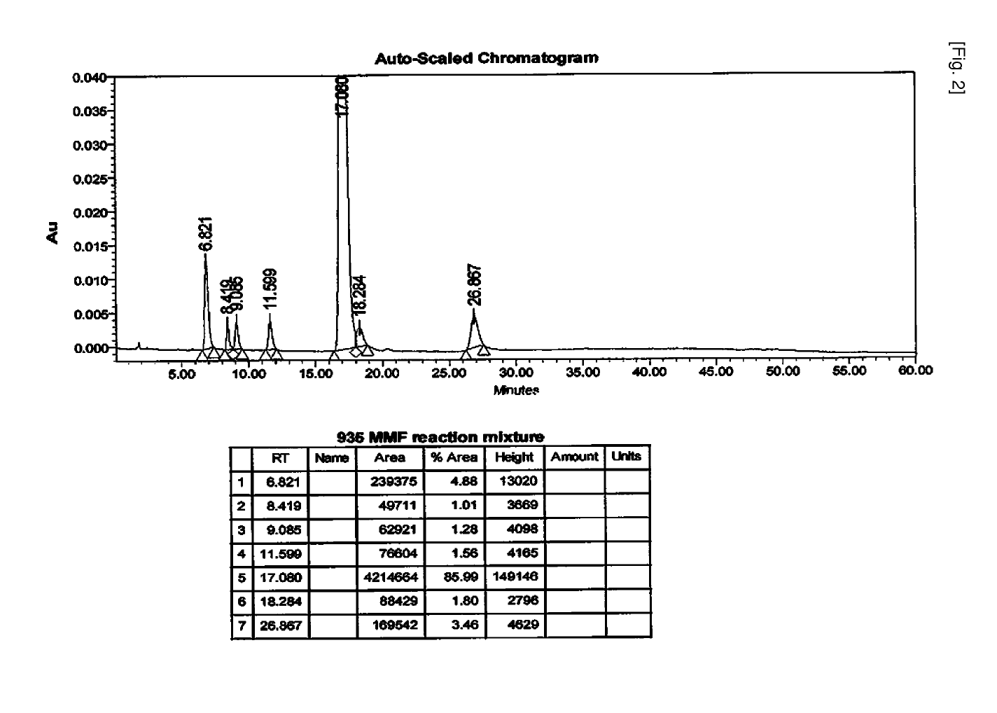

FIGS. 1 and 2 show the respective results of HPLC for the reaction mixture used in the manufacture of MMF according to the present invention by using an amine salt of mycophenolate as an intermediate and the reaction mixture used in the manufacture of MMF according to a method disclosed in U.S. Pat. No. 4,753,935. The effects on the production of an amine salt of mycophenolate based on the HPLC results of FIGS. 1 and 2 were compared and are shown in the Table 1 below.

TABLE 1

| | Mycophenolate | Unreacted | Dimers & Other |
|---|---|---|---|
| Category | Mofetil(MMF) | Mycophenolate | Impurities |
| Reaction mixture | 99.87% | 0.13% | 0% |
| of the present | | | |
| invention | | | |
| Reaction mixture | 85.99% | 4.88% | 9.13% |
| of U.S. Pat. No. | | | |
| 4,753,935 | | | |

The manufacturing method of the present invention is described in further detail as shown below.

The method of manufacturing mycophenolate mofetil represented by the above formula 1 may be performed through 3 steps. Therefore, the manufacturing method of the present invention may be performed by separation and purification of each compound produced in each step, but it is preferred that the 3-step process be preformed continuously as one pot reaction. The details of the manufacturing method of the present invention in each step may be explained as follows.

First, mycophenolic acid represented by the formula 2 below is reacted with an amine base to obtain an amine salt of mycophenolic acid represented by the formula 3 below,

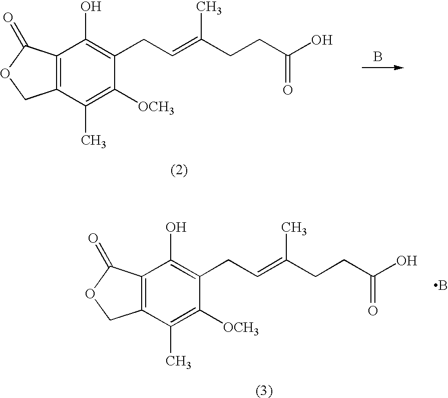

wherein B represents a C₁₋₁₂ aliphatic or aromatic amine base forming a quarternary amine salt.

Then, an amine salt of mycophenolic acid represented by the following formula 3 is halogenated to produce a mycophenolic acid halide represented by the following formula 4.

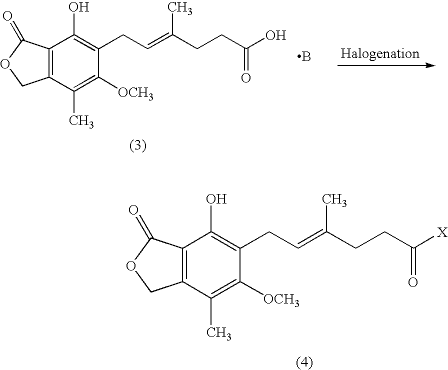

In the above reaction, B represents a C₁₋₁₂ aliphatic or aromatic amine base forming a quarternary amine salt, and X represents a halogen atom.

Then, the mycophenolic acid halide represented by the following formula 4 is esterified by reacting with 2-morpholinoethanol represented by the following formula 5 to obtain mycophenolate mofetil represented by the following formula 1.

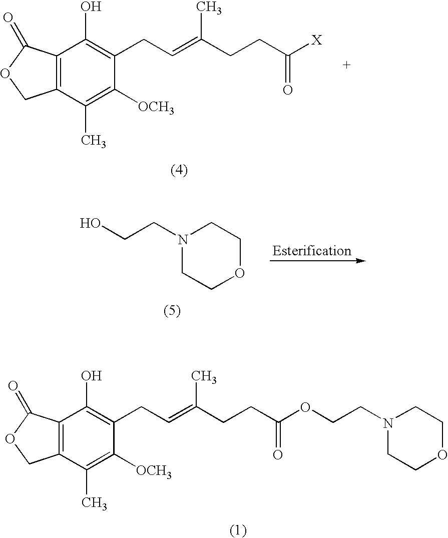

In the above reaction, X represents a halogen atom.

The reaction solvent to be used in the present invention may be any inert solvent which does not affect the reaction. For example, the solvent may be a single solvent selected from the group consisting of: an aromatic hydrocarbon-based solvent such as benzene, toluene, xylene, anisol; an ether-based solvent such as diethylether; an amide-based solvent such as dimethylformamide, diethylacetamide; an acetate-based solvent such as ethyl acetate; a nitrile-based solvent such as acetonitrile; and a halogenated hydrocarbon-based solvent such as chloroform, dichloroethane, dichloromethane; or a mixture thereof.

A preferable reaction solvent is a single solvent selected from ethyl acetate, dichloromethane, toluene, anisol, acetonitriel, 1,4-dioxane; or a mixture thereof; or a solvent comprising these as a main solvent. More preferably, the reaction solvent may be a single solvent selected from ethyl acetate, dichloromethane, anisol; or a mixture thereof; or a a solvent comprising these as a main solvent.

The reaction solvent may be used in the amount of 1-20 volume ratio relative to the amount of total reactants, preferably 5-20 volume ratio, and more preferably 8-12 volume ratio.

The reaction of the manufacturing method according to the present invention can be performed at 0-200° C., preferably at 5-100° C., more preferably at 20 - 60° C., and most preferably at room temperature.

Examples of a chlorinating agent to be used in the present invention include thinoylchloride, oxalylchloride, phosphoruspentachloride, and phosphorusoxychloride; and preferably thinoylchloride, oxalylchloride.

The chlorinating agent may be used in the amount of 0.5-5 equivalents relative to mycophenolic acid represented by the above formula 2, preferably 1-2 equivalents, more preferably 1-1.2 equivalents from the economical point of view.

The amine base to be used in the present invention is preferably a C₁₋₁₂ aliphatic or aromatic amine, and more preferably a single compound selected from aliphatic alkylamine such as triethylamine, diethylamine and aromatic amine such as pyridine, or a mixture thereof.

The amine base may be used in the amount of 0.5-5 equivalents relative to mycophenolic acid represented by the above formula 2, preferably 1-2 equivalents.

The amount of 2-morpholinoethanol used in esterification of the present invention is 1-10 equivalents relative to mycophenolic acid represented by the above formula 2, preferably 1-3 equivalents, and more preferably 1-1.5 equivalents from the economical point of view.

The present invention is also characterized in that it provides a special post-treatment process for the treatment of reactants generated as a result of the above reaction. That is, in the present invention, the processes of acidification and alkalinization, which are performed for the color improvement of mycophenolate mofetil and the removal of other impurities, are performed continuously.

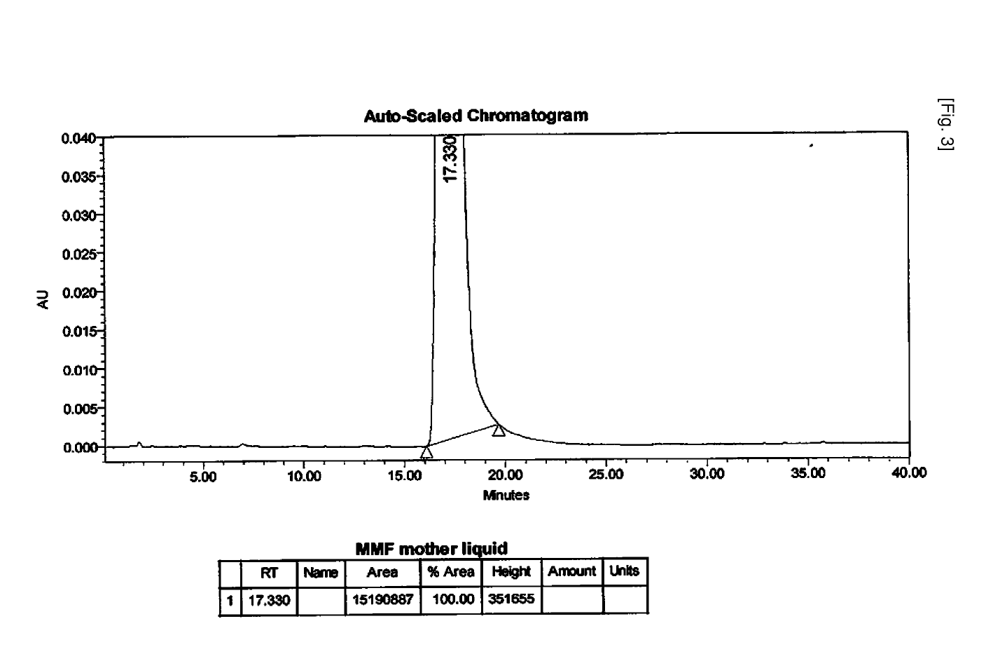

Further, in the above post-treatment process of the present invention, there is added as a discolorant a small amount of alkali metal sulfite-based compound thereby discoloring purple-colored mycophenolate mofetil represented by the above formula 1 in an acidic solution. The above post-treatment has advantages that it can prevent discoloration without additional process by using a small amount of discolorant and ultimately obtain a white-colored compound with high purity. The effects of the post-treatment were confirmed by HPLC results shown in FIG. 3. That is, by comparing the HPLC results for the reaction mixture obtained by the manufacturing method of the present invention as shown in FIG. 1 with those for the white-colored compound obtained by additional post-treatment of the reaction mixture as shown in FIG. 3, the effects of the post-treatment were confirmed. The brief details of the effects of the post-treatment with respect to FIGS. 1 and 3 are shown in Table 2 below.

TABLE 2

| | mycophenolate | unreacted | dimers and other |
|---|---|---|---|
| Category | mofetil(MMF) | mycophenolate | impurities |
| before post- | 99.87% | 0.13% | 0% |
| treatment of | | | |
| reaction | | | |
| mixture of the | | | |
| present | | | |
| invention | | | |
| after post- | 100% | 0% | 0% |
| treatment of | | | |
| reaction | | | |
| mixture of the | | | |
| present | | | |
| invention | | | |

Examples of the discolorants to be used in the present invention include sodium sulfite (Na₂SO₃), sodium metabisulfite (Na₂S₂O₅), sodium hydrogensulfite (NaHSO₃), sodium thiosulfate (Na₂S₂O₃), preferably sodium metabisulfite (Na₂S₂O₅).

The discolorant may be used in the range of 0.05-1 equivalent relative to mycophenolic acid represented by the above formula 2, preferably 0.05-0.15 equivalent.

Examples of acids to be used in the acidification as post-treatment process are hydrochloric acid, phosphoric acid, nitric acid, formic acid, sulfuric acid. The pH range of acidification is in the range of pH 1-4, and preferably pH 1-3.

Examples of alkalis to be used in the acidification as post-treatment process are sodium carbonate, sodium hydrogen carbonate, sodium hydroxide. The pH range of alkalinization is pH 6-10, and preferably pH 7-10.

The following examples illustrate the invention and are not intended to limit the same.

### EXAMPLES

Example 1. Continuous Synthesis 1 of Mycophenolate Mofetil

To 100 g of mycophenolic acid were added 1 L of ethyl acetate and 44 g of triethylamine and the mixture was stirred for about 30 minutes. Then, 44 g of thionyl chloride was dropwisely added and stirred for about 2 hours. The above reactants were added with 123 g of morpholinoethanol and stirred for more than 2 hours to obtain a brown reaction mixture, for which HPLC was performed and the result is shown in FIG. 1. According to the HPLC results shown in FIG. 1, the content of unreacted mycophenolic acid is less than 0.13%, and the presence of dimers or other impurities was not detected.

To the above brown compound was added distilled water and dropwisely added with hydrochloric acid to adjust its pH to below pH 3 and then layer separation was performed. The resulting aqueous layer was added with a small amount of sodium metasulfite and stirred. The resultant was added with 1 L of ethyl acetate and then with sodium carbonate to adjust its pH to higher than pH 7 and then stirred to obtain light yellow organic layer by layer separation. Thus obtained organic layer was dried by using anhydrous magnesium sulfate and filtrated and the resulting filtrate was concentrated under reduced pressure. The resultant was crystallized by using 0.5 L of isopropyl alcohol, filtrated, washed with a small amount of isopropyl alcohol and then dried under vacuum for more than 12 hours to obtain 124 g of mycophenolate mofetil in white powder.

HPLC was performed for thus obtained white-powdered mycophenolate mofetil and the result is shown in FIG. 3.

HPLC analysis: 99.9% or higher of purity, 0% of unreacted mycophenolic acid, presence of dimers and other impurities not detected.

Example 2. Continuous Synthesis 2 of Mycophenolate Mofetil

Experiments were performed same as in the above Example 1 except that pyridine was used as an amine base thereby obtaining 115 g of white-colored mycophenolate mofetil.

HPLC analysis: 99.9% or higher or of purity, less than 0.1% of mycophenolic acid, presence of dimers and other impurities not detected.

Example 3. Continuous Synthesis 3 of Mycophenolate Mofetil

Experiments were performed same as in the above Example 1 except that diethylamine was used as an amine base thereby obtaining 113 g of white mycophenolate mofetil.

HPLC analysis: 99.8% or higher of purity, less than 0.1% of mycophenolic acid, less than 0.1% of other impurities but presence of dimers not detected.

Example 4. Continuous Synthesis 4 of Mycophenolate Mofetil

Experiments were performed same as in the above Example 1 except that dichloromethane was used as a reaction solvent thereby obtaining 123 g of white mycophenolate mofetil.

HPLC analysis: 99.9% or higher of purity, less than 0.1% of mycophenolic acid, prebsence of dimers and other impurities not detected.

Example 5. Continuous Synthesis 5 of Mycophenolate Mofetil

Experiments were performed same as in the above Example 1 except that anisol was used as a reaction solvent thereby obtaining 120 g of white mycophenolate mofetil.

HPLC analysis: 99.9% or higher of purity, less than 0.1% of mycophenolic acid, presence of dimers and other impurities not detected.

Example 6. Continuous Synthesis 6 of Mycophenolate Mofetil

Experiments were performed same as in the above Example 1 except that phosphoric acid was used as an acid in the acidification during the treatment of reactants thereby obtaining 124 g of white mycophenolate mofetil.

HPLC analysis: 99.9% or higher of purity, less than 0.1% of mycophenolic acid, presence of dimers and other impurities not detected.

Example 7. Stepwise Synthesis of Mycophenolate Mofetil

1) Synthesis of mycophenolic acid triethylamine

To 100 g of mycophenolic acid were added 1 L of ethyl acetate and then 44 g of triethylamine. After mycophenolic acid was dissolved, and became white powder, it was stirred for about 30 minutes. The reaction mixture was filtrated, washed with a small amount of ethyl acetate and then dried under vacuum for more than 12 hours to obtain 118 g of mycophenolic acid triethylamine in white powder.

Melting point: 125° C-126° C.; ¹H NMR(CDCl_{3,} 400 MHz) δ1.25(t, 9H, TEA -CH₃), 1.78(s, 3H, -CHC(CH₃)-), 2.13(s, -CH₃), 2.30(s, 4H, -CH₂CH₂CO₂H), 2.87(q, 6H, TEA -CH₂), 3.35-3.37(d, 2H, ArCH₂CH-), 3.74(t, -OCH₃), 5.18-5.23(m, 3H, lacton 2H and -CHC(CH₃)-); HPLC analysis: 98% or higher of purity

2) Synthesis of mycophenolate mofetil

To 118 g of mycophenolic acid triethylamine was added 1 L of ethyl acetate and then dropwisely added with 44 g of thionly chloride and stirred for about 2 hours. To the above reactants was added 123 g of 2-morpholinoethanol and then stirred for about 2 hours to obtain brown reaction mixture. Then, the resultant was purified same as in the above Example 1 and finally 97 g of white-colored mycophenolate mofetil was obtained.

HPLC analysis: 99.8% or higher of purity, less than 0.1% of mycophenolic acid, presence of dimers and other impurities not detected.

Comparative Example 1. Synthesis of Mycophenolate Mofetil by a Conventional Method (disclosed in U.S. Pat. No. 4,753,935)

Mycophenolic acid was dissolved in dichloromethane and then added with thionyl chloride and dimethyl formamide. The reaction mixture was stirred for 3 hours at room temperature and the volatile components were removed under vacuum and then mycophenolic acid chloride was obtained in an oily state. Thus obtained oily mycophenolic acid chloride was dissolved in dichloromethane, cooled down and then added with 2-morpholinoethanol, which was dissolved in dichloromethane and cooled down, and then the mixture was stirred for more than 4 hours at 4° C. to obtain a brown reaction mixture solution. The reaction mixture solution was analyzed by HPLC and the result is shown in FIG. 2. According to FIG. 2, mycophenolate mofetil had 85.99% of purity, the content of unreacted mycophenolic acid was 4.88%, and a total 9.13% of dimers or other impurities were detected.

The reaction mixture was washed with water and then washed again with aqueous sodium carbonate. The organic phase was dried using sodium sulfate and then evaporated to obtain purple mycophenolate mofetil. Thus obtained mycophenolate mofetil, as a result of HPLC analysis, was found to have 97.2% of purity and contain about 1.5% of dimers.

As explained above, the inventors of the present invention succeeded in providing a method for manufacturing mycophenolate mofetil represented by the above formula 1 with high purity to be industrially applicable and economical by reacting mycophenolic acid, which is used as a starting material, with an amine base to obtain a novel amine salt of mycophenolic acid represented by the above formula 3, as an intermediate, thereby significantly inhibiting the production of dimers and other impurities, byproducts of halogenation which have been difficult to remove.

In particular, the process for removing excess chlorinating agent by evaporation under reduced pressure in a highly acidic condition, which is used during halogenation, is no more necessary, and with this procedural advantage, the entire series of manufacturing process is proceeded with in one-pot reaction not necessitating additional process, thereby simplifying the manufacturing process. Further, the entire process time of the manufacturing method of the present invention is much reduced to 2-6 hours as compared to the long process time in the conventional methods, thereby reducing production cost and increasing industrial applicability.

Besides, in the present invention, there is added a small amount of alkali metal sulfite-based compound for color improvement of mycophenolate mofetil represented by the above formula 1 during the treatment of reactants, without additional discoloring process thereby obtaining a white-colored compound. Further, the manufacturing method of the present invention can almost completely remove a small amount of other impurities produced during the above manufacturing process and thus can obtain the mycophenolate mofetil represented by the above formula 1 with purity of 99.8% or higher.

### INDUSTRIAL APPLICABILITY

As mentioned above, the manufacturing method of the present invention has advantages of producing white-colored mycophenolate mofetil (MMF) with high purity and high yield not necessitating additional purification process and is thus expected to be suitable for mass production and industrial application.

1. A method of manufacturing mycophenolate mofetil comprising: (a) synthesizing an amine salt of mycophenolate represented by the Formula 3 below by reacting mycophenolic acid represented by the Formula 2 below with a C₁-C₁₂ aliphatic or aromatic amine base; and (b) manufacturing mycophenolate mofetil represented by the Formula 1 below by halogenation of the amine salt of mycophenolate represented by the Formula 3 below and esterification with 2-morpholinoethanol 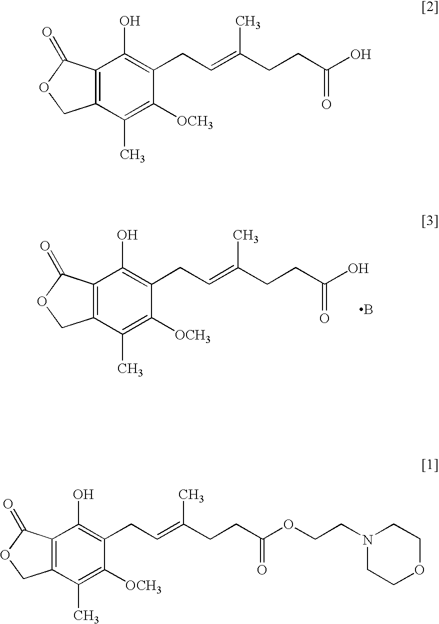
wherein in the above Formula 3, B is an aliphatic or aromatic amine base.

2. The method of manufacturing mycophenolate mofetil according to claim 1, wherein the manufacture of mycophenolate mofetil is performed continuously as one pot reaction without purification of the amine salt of mycophenolate represented by the Formula 3 above.

3. The method of manufacturing mycophenolate mofetil according to claim 1, wherein said amine base is triethylamine.

4. The method of manufacturing mycophenolate mofetil according to claim 1, wherein the amine base is used in the amount of 1 -2 equivalents relative to the amount of mycophenolate represented by the above Formula 2.

5. The method of manufacturing mycophenolate mofetil according to claim 1, wherein the solvent used for the reaction is a single solvent selected from the group consisting of ethyl acetate, dichloromethane, and anisol, or a mixed solvent thereof.

6. The method of manufacturing mycophenolate mofetil according to claim 1, wherein the reaction is performed at 20-60° C.

7. The method of manufacturing mycophenolate mofetil according to claim 1, wherein mycophenolate mofetil with more than 99.8% purity in white color represented by the above Formula 1 is obtained by continuously performing the post-treatment of acidification and alkalinization of the reaction mixture.

8. The method of manufacturing mycophenolate mofetil according to claim 7, wherein said acidification as post-treatment is performed by adjusting the pH of the reaction mixture to pH 1-3 by using hydrochloric acid of phosphoric acid.

9. The method of manufacturing mycophenolate mofetil according to claim 7, wherein said acidified reactant is treated further with Na₂S₂O₅, a discoloring agent, to prevent color change.

10. The method of manufacturing mycophenolate mofetil according to claim 9, wherein said discoloring agent is used in the amount of 0.05 l equivalent relative to mycophenolate represented by the above Formula 2.

11. An amine salt of mycophenolate represented by the following Formula 3: 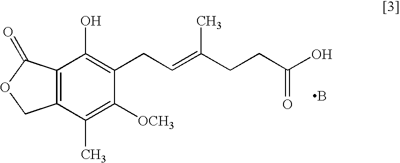
wherein B represents a C₁ C₁₂ aliphatic or aromatic amine which forms a quarternary amine salt.

12. A triethylamine salt of mycophenolate represented by the following Formula 3a.

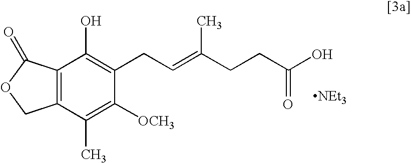

13. The method of manufacturing mycophenolate mofetil according to claim 3, wherein the amine base is used in the amount of 1-2 equivalents relative to the amount of mycophenolate represented by the above Formula 2.

14. The method of manufacturing mycophenolate mofetil according to claim 8, wherein said acidified reactant is treated further with Na₂S₂O₅, a discoloring agent, to prevent color change.
